(19) United States
(12) Patent Application Publication
(10) Pub. No.: US 2021/0228092 A1
(43) Pub. Date: Jul. 29, 2021

(54) TREATMENT RECOMMENDATION CREATION SYSTEM

(71) Applicant: CEIBA TELE ICU SAGLIK HIZMETLERI VE TICARET ANONIM SIRKETI, Istanbul (TR)

(72) Inventors: Afsin ALP, Istanbul (TR); Afsar ALP, Istanbul (TR)

(21) Appl. No.: 17/227,269

(22) Filed: Apr. 9, 2021

Related U.S. Application Data
(63) Continuation of application No. PCT/TR2019/050847, filed on Oct. 9, 2019.

(30) Foreign Application Priority Data
Oct. 9, 2018 (TR) 2018/14873

(51) Int. Cl. A61B 5/0205; A61B 5/021; A61B 5/024; A61B 5/145; A61B 5/00; A61B 5/091; G08B 5/22; G16H 40/67; G16H 10/60; G16H 20/40; G16H 10/40; G16H 15/00; G16H 80/00

(52) U.S. Cl. CPC A61B 5/0205; G16H 80/00; A61B 5/024; A61B 5/14542; A61B 5/746; A61B 5/486; A61B 5/091; A61B 5/7475; A61B 5/4848; A61B 5/7405; G08B 5/22; G16H 40/67; G16H 10/60; G16H 20/40; G16H 10/40; G16H 15/00; A61B 5/021

(57) ABSTRACT

A system (1) capable of generating an alert for customized parameters and upon reaching predefined limits for the patient, as per the patient's medical condition, diagnosis and current vital data and designing the alerts and sending them to relevant persons to automatically recreate, update or continue the treatment order which is designed according to the previous alert in the system (1).

This application is a continuation of the International Patent Application No. PCT/TR2019/050847 filed on Oct. 9, 2019, which claims a priority of the Turkish Patent Application No. 2018/14873, filed on Oct. 9, 2018, the contents of each of which are hereby incorporated by reference.

### TECHNICAL FIELD OF THE INVENTION

This invention relates to a system for creating treatment recommendations.

### PRIOR ART

In intensive care units, status of patients are monitored and their treatment sets may be changed when certain parameters change. Although the patient's treatment is determined by the attending physician, certain parameters are set by some devices in the state of the art.

In the state of the art, the alerts for patients in intensive care are viewed via medical devices in the healthcare industry. Such devices do not generate alerts in real time according to patient-specific data, thus the physicians may delay in determining the applicable treatment method and medication parameters. Orders placed with standard ordering methods occasionally lead to delays as physicians are not able monitor every patient at all times. Delayed orders may slow down the patient treatment process. A system to support the physician's diagnosis and treatment is vital to the intensive care units where every second is crucial.

One of the patient care systems utilized in the state of the art is defined in the patent document US2004143171. This document defines a system capable of comparing patient's health condition 20 and data while under medication with predefined standard medical practices using the data provided by patient to check if the medication is administered in the right type and dosage compared to such standards, and to check any side effects of medication. However, this system operates on the basis of patient-provided data and recommends standard medical practices, thus it is not suitable to assist the ICU physician in diagnosing and treating the patient's illness and problem at that time.

Another document in the state of the art, patent document US2014379366 (Al) specifies a system that selects and recommends one of the available treatment plans depending on the severity of the patient's condition. This system is also not suitable for assisting ICU physicians and boosting the speed and accuracy of treatment. Therefore, in current systems, constant monitoring of patient by the attending physician/nurse is required to switch to a treatment more suitable for the patient. This may lead to delays and even omission in initiating new treatments.

### BRIEF DESCRIPTION OF THE INVENTION

The purpose of this invention is to create a unique alert for each patient based on instant patient data.

Another purpose of the device is to establish a system recommending a treatment set customized for each patient based on the aforesaid alerts (the data entered into the system for the patient).

Another purpose of this invention is to ensure that the alert and order created by the physician for the patient is automatically issued by the system, if the treatment recommended by the system is not accepted.

The system in this invention generates alerts or outputs at specific parameters customized for each patient based on the patient's medical condition, diagnosis and current vital data. Based on previous patient parameters, the system designs the alerts or outputs for the alert set by the physician on the system and automatically creates a treatment order previously designed within the respective alert upon reaching specific parameters in patient data. The physician creates an order by using the treatment set recommended by the system or by preparing one himself. System connects the order with an alert. When an alert is given, the order is assigned to the patient and can be directed to the respective medical staff.

### DETAILED DESCRIPTION OF THE INVENTION

### Description of Figures

FIG. 1: Schematic block diagram of the invented system's application.

FIG. 2: Flow diagram showing steps of the treatment selection recommendation method implemented by the system.

FIG. 3: Flow diagram showing steps of the sensitivity analysis method implemented by the system.

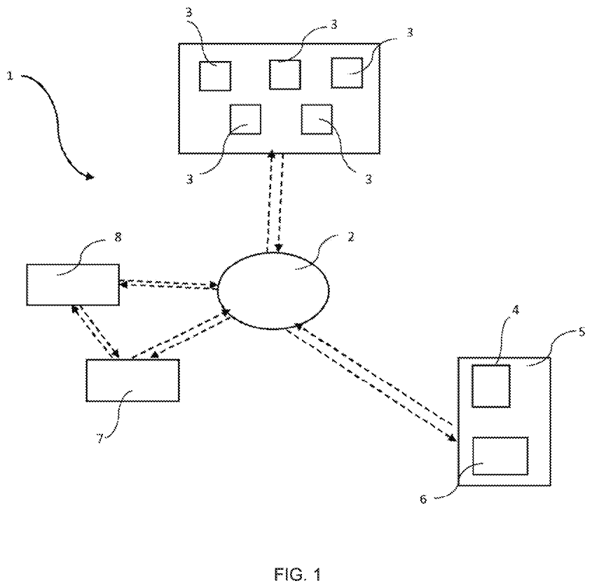

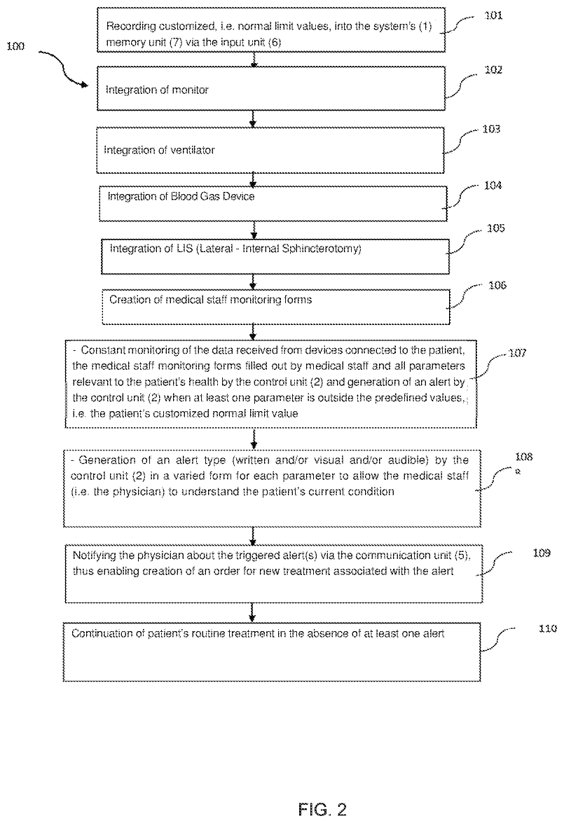

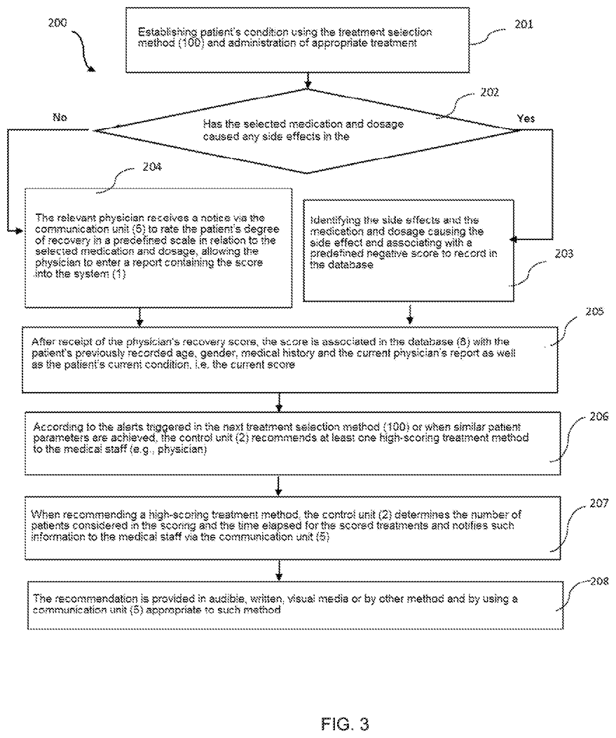

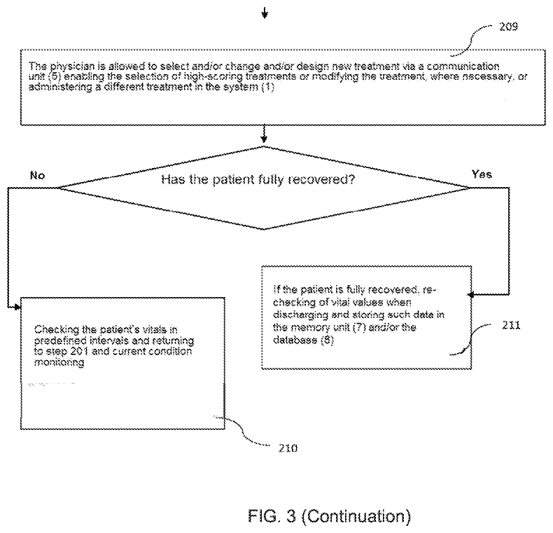

### DESCRIPTION OF REFERENCES IN THE FIGURES

For comprehensibility of the invention, the parts in the attached figures have been individually numbered with corresponding definitions provided below.
1. System
2. Control unit
3. Connected unit
4. Alert unit
5. Communication unit
6. Input unit
7. Memory unit
8. Database
100. Treatment selection recommendation method
200. Sensitivity analysis method

The system (1) comprises a control unit (2) to check the applicable steps in the system (1) and to make decisions, at least one connected unit (3) to connect to the patient and receive instant data, an alert unit (4), a communication unit (5) to transmit system (1) outputs to relevant persons or units, an input unit (6) for medical staff to enter predefined patient data, a memory unit (7) to store the entered data and a database (8) to associate with each other the data provided.

The connected unit (3) refers to devices providing all information such as monitor, ventilator, pump, laboratory, blood gas and demographic information.

The alert unit (4) is a module generating patient-specific alerts. It generates an alert when one or more parameters are outside the predefined values. The alert unit (4) provides differentiated alerts for each parameter. This allows the medical staff (such as physician) to easily comprehend the patient's current problem. The alerts may be issued in written and/or visual and/or audible form. The alert unit (4) may contain modules depending on the invention's application, such as a module for SMS transmission, a screen or a pager.

The communication unit (5) allows the medical staff to be informed about alert(s). The communication unit (5) is configured to allow the medical staff to select a treatment method, to make changes to the treatment method or to apply a different treatment when necessary, in line with the relevant alert.

The communication unit (5) contains user interface and or graphical user interface (GUI) to allow user input. In one application of the invention, the communication unit (5) contains the input (6) and alert units (4). The database (8) can be internal or external to the memory unit (7) depending on the invention's applications. The invention predefines the alert types in the memory unit (7) according to alert severity and triggering parameters and the respective parameters and alert types are associated in the database (8). Depending on the extent of the exceeded predefined patient limits and on which patient parameter is exceeded, the alert types are predefined in the memory unit (7) and the data and alert types are associated in the database (8). Each patient's individual patient limits and individual exceeding values (i.e. severity) and small and big exceeding values are predefined and recorded in the memory unit (7) and this data is associated with each other in the database (8).

The medical staff, as defined in this description document, refers to physicians, nurses and other persons authorized to monitor and administer patient treatment process.

All actions, records, decisions, comparisons and assessments mentioned as carried out by the system (1) in this document are carried out by the control unit (2) included in the system (1).

All decisions taken as a result of the assessments carried out by the control unit (2) of the system in this description, all comparisons and assessments are notified to medical staff via the communication unit (5).

In this description, the treatment method, referred to as treatment herein, means antibiotics, etc. medicine use and its dosage, while treatment set means stomach soother, cream, antibiotics, nutrition (food), antihypertensive, antiarrhythmic medication, drugs used by patient or recommended by physician as well as various substances and dosages used by patient and their administration.

The system (1) implements the following treatment selection recommendation method (100) steps:
Recording in the memory unit (7) of the system (1) of the patient-specific values, i.e. the normal limit values of the patient such as blood pressure, heart rate (HR) systolic arterial pressure (SAP), etc., which are different for each person, via entering these values with the input unit (6) (101),
Providing monitor integration via instant transfer of all patient monitor data, HR (heart rate), SAP (Systolic Arterial Pressure), MAP (Mean Arterial Pressure), DAP (Diastolic Arterial Pressure), etc.) to the system (1) (102),
Providing ventilator integration via instant transfer of patient's all ventilator data (i.e. Oxygen, FiO2 (Fraction of inspired Oxygen), Peep (Positive End Expiratory Pressure), etc.) to the system (1) and recording in system (1) memory unit (7) (103),
Providing (104) Blood Gas Device integration via transmission of all blood gas data (i.e. pO2 (partial pressure of oxygen), pCO2 (partial pressure of carbon dioxide), etc.) of the patient to the system (1) instantly upon completion of measurement and recording in the memory unit (7) in case of blood gas is measured (104),
Providing integration of LIS (Laboratory-Internal Sphincterotomy) via transfering of all laboratory data (i.e. ALT (Alanine Aminotransferase), APTT (Activated Partial Thromboplastin Time), CRP (C-Reactive Protein) etc.) of the patient to the system (1) instantly upon attainment of laboratory results and recording in the system (1) memory (7) (105),
Ensuring the medical staff attending the patient (i.e. nurse) to fill out the forms with the data comprising the patient's current condition, neurological evaluation, pain management, infection, extracorporeal, wound care, physical examination findings via utilizing the input unit (6) to enter such data in the system (1) and recording of such forms in the system (1) memory (7), thus creating the medical staff monitoring forms (106),
Continuously controlling of the data received from devices connected to the patient, the medical staff monitoring forms filled out by medical staff and all parameters relevant to the patient's health by the control unit (2) and generation of an alert by the control unit (2) when at least one parameter is outside the predefined values, i.e., the normal limit values specific to the patient (107),
Generation of an alert type (written and/or visual and/or audible) by the control unit (2) in a varied form for each parameter to allow the medical staff (i.e. the physician) to understand the patient's current condition (108),
Notifying the physician of current alert(s) through the communication unit (5) to ensure an order for a new treatment relevant to the alert is created and therefore, instant updating of treatment regimen via generation of instant alert or alerts is provided and so, implementation of new treatment method (i.e. treatment or treatment set) is provided (109),
Continuation of patient's routine treatment in the absence of at least one alert (110).
   In step 107 when the routine treatment method or new treatment method are determined, the control unit places on account, i.e. considers all the parameters of the patient's age, gender, medical history and current condition, i.e. current parameters, anamnesis and epic risis details and previous alert(s), which are included in the calculations. The anamnesis and epicrisis information mentioned here are as follows: the physician checks the anamnesis after acceptance of patient to Intensive Care. The anamnesis contains information on past and current medication, history of surgeries and disorders, background, family history (i.e. genetic), etc. The anamnesis for the medical staff attending the patient, for instance nurse contains information such as the dosages of medication administered to the patient. Finally, the epicrisis contains information on the patient's initial complaints, medication used, examination (blood, graphy, CT, MRI, etc.) results.

In one application of the invention, the patient's normal values have been set as for example HR (Heart Rate)=120, SAP (Systolic Arterial Pressure),=70, temperature=37.8 and pH=7.25. In this application, the control unit (2) creates an alert when at least one of these values is outside the aforesaid values. Ex: HR>120 and/or SAP<70 and/or Temperature>37.8 and/or pH<7.25 measurement triggers the alert and an information message is sent to the attending physician. The control unit (2) continuously checks the forms recorded by medical staff and measurements of devices connected to the patient.

The system (1) contains the customized limit values for each person recorded in the memory unit (7) based on the patient's history. This allows the alerts to be specific to the patient's history and diagnosis, i.e., customized to the patient.

In the system (1), alert types for the alert severity and associated parameter are predefined. For instance, alert types are predefined according to the extent of exceeding the predefined patient limits, being small or big, and according to which parameter exceeds normal limit values. The small or big limit exceeding of patient limits may vary for each patient and they are also predefined and recorded in the memory unit (7).

The invention associates a treatment method for each alert type and records it in advance in the database (8). Thus, registered treatment methods can be automatically administered depending on the respective alert for quicker response. This is recorded in the memory unit (7) as an automatic order.—This occurs in the following manner: The physician defines which treatment(s) are to be administered when patient parameter values change in the database (8). Each of these definitions are physician's orders. These orders have been associated with possible alerts. The Control Unit (2) checks for any patient-specific alerts and any orders associated with such alerts. If an alert is generated that is associated with an order, the control unit (2) assigns the order to the patient upon generation of the alert and the medical staff (e.g. nurses) administer the order. This ensures saving time spent on comparison of all parameters with predefined criteria, the comparison is carried out by the system (1), thus minimizing the error margin, preventing nurses and physicians from omitting or missing defined criteria, the system (1) maintains instant controls, thus the new treatment is engaged without delay.

The invented system (1) recommends treatment or treatment sets to intensive care patients depending on data (patient's limit values, patient's diagnosis, nurse, physician reports, etc.) recorded in the system (1). The invention records all patient data, including treatment procedures, medication received, all evaluations of the patient, laboratory results, etc. in the database (8). This enables calculation of the success rate administered to a specific patient profile. The control unit (2) establishes the individual success rate for each treatment administered. Percentage of success increase in proportion to the number of samples. The success rates and percentages are recorded by the control unit (2) associated with the treatment(s) administered in the database (8). When a similar patient with a similar profile is received and the specific alert limits defined for the patient are attained, the most suitable order will be the one with the highest rate of success with past patients, which will be automatically recommended by the control unit (2).

When recommending such substances, the control unit (2) also transmits to the medical staff the dosage/amounts to be administered, when and how to use the substance (e.g. when hungry, full, morning, evening, etc.) via the communication unit (5).

The treatment and treatment set contains information on the dosage and/or amounts of substances 5 to be administered to the patient, the time and method (e.g. hungry, full, morning-evening etc.) of administration.

When establishing the treatment or treatment set, the control unit (2) takes into account the patient's age, gender, medical history and current condition.

A sensitivity analysis method (200) is also managed by the invented system (1). In this method (200), upon selection of treatment using the treatment selection method (100) specified above and its administration, the treatment method is scored by the control unit (2). The analysis is carried out as follows: The control unit (2) checks the patient's condition before and after administration of medication in regular intervals and creates the sensitivity score analysis.

The sensitivity analysis method (200) operates as follows:
The patient's condition is established (i.e. condition analyzed) using the treatment selection method (100) and appropriate treatment is administered (201),
Checking for any side effects of the selected medication and its dosage in the patient after administration (202),
If any side effects are present, identification of the side effects and recording the medication and dosage causing this side effect with a predefined negative score in the database (8) (203),
If the selected medication and dosage had no side effects, sending the relevant physician a notice via the communication unit (5) to rate the patient's degree of recovery in a predefined scale in relation to the selected medication and dosage, allowing the physician to enter a report containing the score into the system (1) (204),
Following receipt of the physician's recovery score, associating the patient's previously recorded age, gender, previous diseases and current physician's report with the patient's current condition in the database (8) (205),
Depending on the alerts created in the next treatment selection method (100) and if similar patient parameters are achieved (e.g. two of the following parameters: “are the new patient's history of illnesses the same as or similar to the previous patient's history”, “is the age or gender the same or similar”), the control unit (2) recommends at least one high-scoring treatment method to the medical staff (e.g., physician) (206),
When recommending a high-scoring treatment method, the control unit (2) determines the number of patients considered in the scoring and the time elapsed for the scored treatments and notifies such information to the medical staff via the communication unit (5) (207),
The recommendation is provided in audible, written, visual media or by other method and by using a communication unit (5) suitable forto such method (208),
The physician is allowed to select and/or change and/or design a new treatment via a communication unit (5) configured in such a way that it enabling the selection of high-scoring treatments or modification of the treatment, where necessary, or administration of a different treatment in the system (1) (209),
Re-checking of the patient's vital data in predefined periods and returning to step 201 for last status monitoring (210),
If the patient is fully recovered, re-checking of vital values when discharging and storing such data in the memory unit (7) and/or the database (8) (211).

In order to establish the percentage of similarity in step 207: The medical parameters to be compared and the low and high limits of such parameter for the concerned patient as well as the degree of similarity of percentages are pre-assigned in the database (8). The control unit (2) checks such associations for the previous (previously recorded) and new patient, establishing the percentage of similarity between the previous and the current patient.

In a preferred application of the invention, the treatment methods of previous (former) patients are displayed (i.e., recommended) in a degree of similarity in descending order of percentage to the physician via a communication unit (5) such as a screen. In this step, the dissimilar parameters of patients with even highly similar disorders and with similar low and high parameter limits, such as 90%, are notified to the attending physician, as the slightest variation may change the course of treatment and may be fatal in a sensitive environment, such as the ICU, thus all data, including dissimilar parameters, are notified to the physician by the control unit (2) for the physician evaluation.

The control unit (2) scores the success rate of the treatment using the sensitivity analysis method (200). Using this analysis, the control unit (2) learns continuously new treatments and recommends a new treatment to the physician for the next treatment selection method (100) (i.e., a treatment or treatment set). This allows the physician to select the high-scoring treatment method in the system (1) for similar patients or make changes to the treatment or to administer a different treatment.

Another advantage of the sensitivity analysis method (200) is that it shows the high-scoring medication for a specific illness, enabling analysis of which active contents are effective on that illness. For this purpose, the illnesses and medications that may be used for such illness, as well as the active agents of such medication have been pre-defined and associated with each other in the database (8).

In the invention, it also records in the database (8), the results of specific treatments for individual patients through demanding the physician to fill a report and in a way to associates the results with the relevant illnesses. This will enable the recorded treatment results to be used in research and development activities.

The invented system (1) to be used for intensive care patients will allow automatic receipt of all data in the system (1). This enables any changes in the parameters to be instantly detected by the system (1).

The system (1) features a control unit (2) which generates an alert when any medical parameter is outside the patient's own normal limit value, thus an alert unique to the patient is created by using multiple parameters and the system (1) recommends at least one treatment method (treatment or treatment set) customized to the patient via taking into account the customized limits for each patient.

Via automation and integration in the intensive care unit, the system (1) continuously receives all patient data from monitors, ventilator, pump, laboratory, blood gas and demographics. Nurse monitoring forms, physician's notes, orders and other actions are carried out via the system (1). This will ensure that an order issued by the physician with criteria set in the system (1) will utilize all available data and result in faster and more accurate decisions.

In another application of the invention a “control tower” sends information on the patient's condition (septic shock and other specific cases) to the medical staff assigned to the patient's care, such as physicians and nurses via sms/e-mail, etc. The communication unit carries out this function. The physician specifies priority patients or isolation rooms to receive priority information from such patients. This is realized by that the communication unit sends out an alert with a different code.

In another application of the invention based on the daily data of intensive care patients, the physician's notes can be created by around 80%. The system (1) in question draws data from the patient's vital, respiratory, laboratory, blood gas results, medical devices and the integrated Hospital Information Management Systems.

Such data and the fields completed by the physician in multiple-choice form will be structured into sentences by the control unit (2) to create the epicrisis report. As an example, the patient's vital values are SAP=120, TEMPERATURE=37, respiratory value VTE=540, FiO2=60 and laboratory values are Albumin=3.4. The physician for example selects the multiple-choice Step 1 as Invasive, Enteral. For this instance, the system will create the sentence: “The Patient received Step 1 intensive care. Invasive mechanical ventilation support is administered. Vital values are: SAP=120, Temperature=37. Respiratory values are VTE=540, FiO2=60. Laboratory values are Albumin=3.4.” The physician may add to the notes using the communication unit (5) and completes the note draft. Additionally, integration with the Hospital Information Management System allows the hospital's laboratory, demographics, order details, allowing the control unit (2) to combine such data with the apache scoring (Acute Physiological and Chronic Health Evaluation) and the physician's diagnosis to create the epicrisis report with time stamp and e-signature (in pdf). An epicrisis report prepared in this form with the physician's signature is completed and can be sent to the insurance company.

The invention is not limited to the above-mentioned practices and a person with technical expertise may easily reveal various applications for the invention. These should be considered within the scope of the invention's claims and the requested protection.

1. A treatment recommendation creation system (1) comprising at least one connected unit (3) for connecting to the patient and receiving data, a communication unit (5) for transmitting system (1) outputs to relevant persons or units, an input unit (6) for allowing medical staff to enter predefined information of the patient, a memory unit (7) to store such information, a database (8) to associate such information with other information and a control unit (2) which keeps track of the steps of methods to be applied and, which takes decisions via making comparisons and evaluations, and ensuring implementation of these decisions by other units (3,4,5,6,7,8), such treatment recommendation creation system (1) wherein
the said memory unit (7) where the individual limit values for each patient are predefined according to the patient's history,
said control unit (2) which provides generation of an alert by the alert unit (4) when at least one parameter is outside the predefined relevant value i.e. customized limit values for that individual patient, thus generating a customized i.e. individualized alert depending on the patient history and specific diagnosis of the patient wherein the control unit (2) checks the following parameters before generation of an alert:
checking instantaneously of the monitoring data, which belongs to that patient, and which are received from connected units (3) such as HR (heart rate), SAP (systolic arterial pressure), MAP (Mean arterial pressure), DAP (diastolic arterial pressure),
checking instantaneously of all ventilator data, which belongs to that patient, and which are received from connected units (3), such as Oxygen, FIO2 (Fraction of inspired oxygen), Peep (Positive End Expiratory Pressure),
checking instantaneously of all blood gas data (example: pO2 (partial pressure of oxygen, 20pCO2 (partial pressure of carbon dioxide), belonging to that patient, if blood gas measurement data is received from a connected unit (3),
checking instantaneously of all laboratory data, belonging to that patient, entered into the system (1) (Example: ALT (Alanine aminotransferases), APTT (activated partial thromboplastin time), CRP (C-reactive protein) and LIS (Laboratory-Internal Sphincterotomy),
checking instantaneously of all medical staff reports, belonging to the patient, entered instantaneously in the system (1) following clinical examination of that patient;
monitoring instantaneously of predefined normal limit values that are customized for thate patient with respect to blood pressure, heart rate (HR), systolic artery pressure (SAP), etc. wherein the normal limit values are unique for each patient;
checking instantaneously of all patient parameters;
and also characterized by;
an alert unit (4) generating differentiated alerts from each other for each parameter to allow the medical staff (e.g. physician) to understand the patient's current problem;
and a communication unit (5), informing the medical staff of such alert(s), and configured such that it allows the medical staff to select a method of treatment or change the treatment method or to implement a different treatment in accordance with the respective alerts,
and the above-mentioned control unit (2) thereby instantly updates the course of treatment and ensure the recommendation and execution of a new treatment method (i.e., treatment or treatment set) via such instant alert or alerts.

2. The system (1) according to claim 1, comprising a memory unit (7) where the alert types are predefined according to severity and triggering parameter and a database (8) where the alerts are associated with the parameters in question.

3. The system (1) according in claim 2, comprising a memory unit (7) wherein the alert types are predefined according to minor or great limit exceeding of predefined patient limits and according to which patient parameter exceeds the normal limit values; and a database (8) to associate such information.

4. The system (1) according to claim 2, comprising a memory unit (7) to record the customized limits and exceeding (i.e., severity) values for each patient and the predefined minor and great limit exceeding values and a database (8) to associate such information.

5. The system (1) according to claim 2, comprising a database (8) wherein the respective treatment(s) in response to changing values of patient parameters are defined as a physician's order, and such orders are associated with possible alerts.

6. The system (1) according to claim 5, wherein the control unit (2) checks whether the patient-specific alerts are generated or not and whether any orders are linked for the respective alert and transmits such orders instantly to the patient when an alarm is linked with an order.

7. The system (1) according to claim 1, comprising a control unit (2) which establishes a success score for each treatment administered, which records in the database (8) such success score in relation to an administered treatment or treatments and which recommends the most suitable order as an order that is obtained from the treatment with the highest success rates for former patients, in case of a similar profile patient is faced and if this patient reaches the patient-specific alert limits that is defined for that patient.

8. The system (1) according to claim 7, comprising a control unit (2) which establishes a sensitivity degree analysis via checking the patient's values before and after medication in predetermined regular intervals.

9. The system (1) according to claim 1, comprising a database (8) in which the medical parameters to be compared and their low and high limit values for the respective patient as well as percentiles of which will deemed to be similar at what rate, are associated in advance, and a control unit (2) which elicits the percentage of similarity of the current patient with at least one previous patient via controlling these relations.

10. The system (1) according to claim 1, wherein the control unit (2) transmits (i.e. recommends) to the medical staff, the percentage of similarity of medical parameters (such as previous illnesses and customized low and high limits) of previous (i.e. former) patients via listing the similarity rates in descending orders as well as associated treatment methods with these medical parameters via using a communication unit (5) such as a display.

11. The system (1) according to claim 10, wherein the control unit (2) provides the dissimilar parameters of previous patients as well as similar parameters via a communication unit (5).

12. The system (1) according to claim 11, wherein a communication unit (5) provides notifications such as sms/e-mail to the attending medical staff such as physicians and nurses in the intensive care unit, relating the patient's condition (septic shock, etc.) and provides notice with a different code for patients in emergency.

13. The system (1) according to claim 1, wherein the control unit (2) creates a time-stamped and e-signed epicrisis report via transforming the multiple-choice fields provided to be completed by the physician into sentences wherein the multiple-choice fields completed by the physician via receiving the patient's vital, respiratory, laboratory, blood gas data from medical devices and the daily data—(i.e. laboratory, demographics, order details and apache scoring (Acute Physiological and Chronic Health Evaluation) established by the control unit (2) and the physician's diagnosis)—of the intensive care patient from the integrated Hospital Information Management System, and via the communication unit (5).

14, A treatment selection recommendation method (100), to be implemented in the system (1) comprising the following steps;
Recording in the memory unit (7) of the system (1) of the patient-specific values, i.e. the normal limit values of the patient such as blood pressure, heart rate (HR) systolic arterial pressure (SAP), etc., which are different for each person, via entering these values with the input unit (6) (101),
Providing monitor integration via instant transfer of all patient monitor data, HR (heart rate), SAP (Systolic Arterial Pressure), MAP (Mean Arterial Pressure), DAP (Diastolic Arterial Pressure) etc.) to the system (1) (102),
Providing ventilator integration via instant transfer of patient's all ventilator data (i.e. Oxygen, FiO2 (Fraction of inspired Oxygen), Peep (Positive End Expiratory Pressure), etc.) to the system (1) and recording in system (1) memory unit (7) (103),
Providing (104) Blood Gas Device integration via transmission of all blood gas data (i.e. pO2 (partial pressure of oxygen), pCO2 (partial pressure of carbon dioxide), etc.) of the patient to the system (1) instantly upon completion of measurement and recording in the memory unit (7) in case of blood gas is measured (104),
Providing integration of LIS (Laboratory-Internal Sphincterotomy) via transferring of all laboratory data (i.e. ALT (Alanine Aminotransferase), APTT (Activated Partial Thromboplastin Time), CRP (C-Reactive Protein) etc.) of the patient to the system (1) instantly upon attainment of laboratory results and recording in the system (1) memory (7) (105),
Ensuring the medical staff attending the patient (i.e. nurse) to fill out the forms with the data comprising the patient's current condition, neurological evaluation, pain management, infection, extracorporeal, wound care, physical examination findings via utilizing the input unit (6) to enter such data in the system (1) and recording of such forms in the system (1), memory (7), thus creating the medical staff monitoring forms (106),
Continuously controlling of the data received from devices connected to the patient, the medical staff monitoring forms filled out by medical staff and all parameters relevant to the patient's health by the control unit (2) and generation of an alert by the control unit (2) when at least one parameter is outside the predefined values, i.e., the normal limit values specific to the patient (107),
Generation of an alert type (written and/or visual and/or audible) by the control unit (2) in a varied form for each parameter to allow the medical staff (i.e. the physician) to understand the patient's current condition (108),
Notifying the physician of current alert(s) through the communication unit (5) to ensure an order for a new treatment relevant to the alert is created and therefore, instant updating of treatment regimen via generation of instant alert or alert is provided and so, implementation of new treatment method (i.e. treatment or treatment set) is provided (109),
Continuation of patient's routine treatment in the absence of at least one alert (110).

15. The method according to claim 14, wherein the control unit (2), considers the patient's age, gender, previous diseases and current condition, i.e., current parameters, medical history and epicrisis details and any previously triggered alerts and takes into account all these criterions in calculation when determining routine treatment method or new treatment method in step 107.

16. A sensitivity analysis method (200) to be implemented in a system (1) comprising the following steps:
Analysis of the patient's condition using the selection method (100) and selection and administration of the suitable treatment (201),
Checking for any side effects of the selected medication and its dosage in the patient after administration (202),
If any side effects are present, identification of the side effects, and recording the medication and dosage causing this side effect with a predefined negative score in the database (8) (203),
If the selected medication and dosage had no side effects, sending the relevant physician a notice via the communication unit (5) to rate the patient's degree of recovery in a predefined scale in relation to the selected medication and dosage, allowing the physician to enter a report containing the score into the system (1) (204),
Following receipt of the physician's recovery score, associating the patient's previously recorded age, gender, previous diseases and current physician's report with the patient's current condition in the database (8) (205),
Depending on the alerts created in the next treatment selection method (100) and if similar patient parameters are achieved (e.g. two of the following parameters: “are the new patient's history of illnesses the same as or similar to the previous patient's history”, “is the age or gender the same or similar”), the control unit (2) recommends at least one high-scoring treatment method to the medical staff (e.g., physician) (206),
When recommending a high-scoring treatment method, the control unit (2) determines the number of patients considered in the scoring and the time elapsed for the scored treatments and notifies such information to the medical staff via the communication unit (5) (207),
The recommendation is provided in audible, written, visual media or by other method and by using a communication unit (5) suitable for such method (208),
The physician is allowed to select and/or change and/or design a new treatment via a communication unit (5) configured in such a way that it enables the selection of high-scoring treatments or modification ofying the treatment, where necessary, or administration of a different treatment in the system (1) (209),
Re-checking of the patient's vital data in predefined periods and returning to step 201 for last status monitoring (210),
If the patient is fully recovered, re-checking of vital data before discharge and recording in the memory unit (7) and/or database (8) (211).
